(19) United States
(12) Patent Application Publication
(10) Pub. No.: US 2014/0243208 A1
(43) Pub. Date: Aug. 28, 2014

(54) COMPOSITIONS AND METHODS FOR SELECTING APTAMERS

(71) Applicants: Yie-Hwa Chang, St. Louis, MO (US); Ling Tian, St. Louis, MO (US); Rongsheng Wang, St. Louis, MO (US)

(72) Inventors: Yie-Hwa Chang, St. Louis, MO (US); Ling Tian, St. Louis, MO (US); Rongsheng Wang, St. Louis, MO (US)

(73) Assignee: Mediomics, LLC, St. Louis, MO (US)

(21) Appl. No.: 14/234,329

(22) PCT Filed: Jul. 23, 2012
(86) PCT No.: PCT/US12/47840

§ 371 Date: Feb. 17, 2014

Related U.S. Application Data
(60) Provisional application No. 61/510,796, filed on Jul. 22, 2011.

(51) Int. Cl. C12N 15/10; C12N 15/115

(52) U.S. Cl. CPC C12N 15/1048; C12N 15/115
USPC 506/1; 536/23.1; 530/391.1

(57) ABSTRACT

The invention encompasses compositions and methods for selecting aptamers.

### GOVERNMENTAL RIGHTS

This invention was made with government support under HHSN268201000030C awarded by the National Heart, Lung and Blood Institute. The government has certain rights in the invention.

### FIELD OF THE INVENTION

The invention encompasses compositions and methods for selecting aptamers.

### BACKGROUND OF THE INVENTION

Aptamers are single stranded nucleic acid sequences that can specifically recognize a target molecule. Methods of selecting aptamers that bind to a target are known, but typically are very time consuming and labor extensive. Hence, there is a need in the art for a quick, efficient, and effective method for selecting an aptamer that binds to a particular target.

### SUMMARY OF THE INVENTION

One aspect of the present invention encompasses a composition minimally comprising three constructs. A composition may comprise a bridge construct (H1), at least one aptamer construct (I1-I2-I3-I4), and an epitope binding agent construct (J1-J2-J3). Alternatively, a composition may comprise a bridge construct (H1) and at least two aptamer constructs (I1-I2-I3-I4).

Another aspect of the present invention encompasses a method of using a composition of the invention to select one or more aptamers. In one embodiment, a method of the invention comprises contacting a composition of the invention with a target, separating a stable complex from the reaction mixture, and determining the identity of the aptamer sequence(s) that recognize the target.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

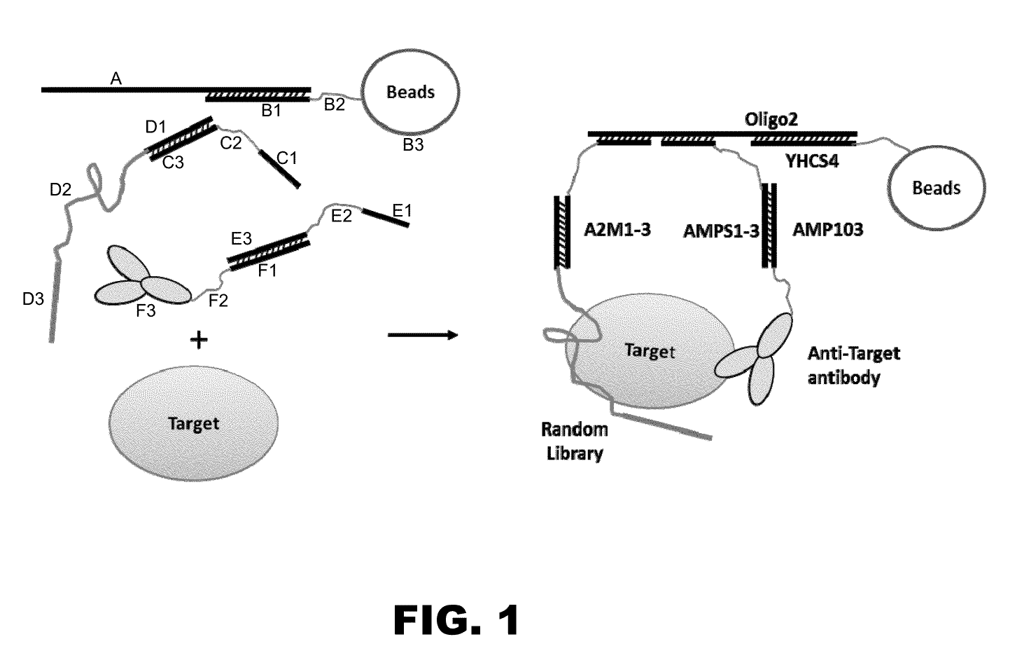

FIG. 1 depicts an illustration showing a composition for selecting aptamers against a target with the assistance of an existing epitope binding agent.

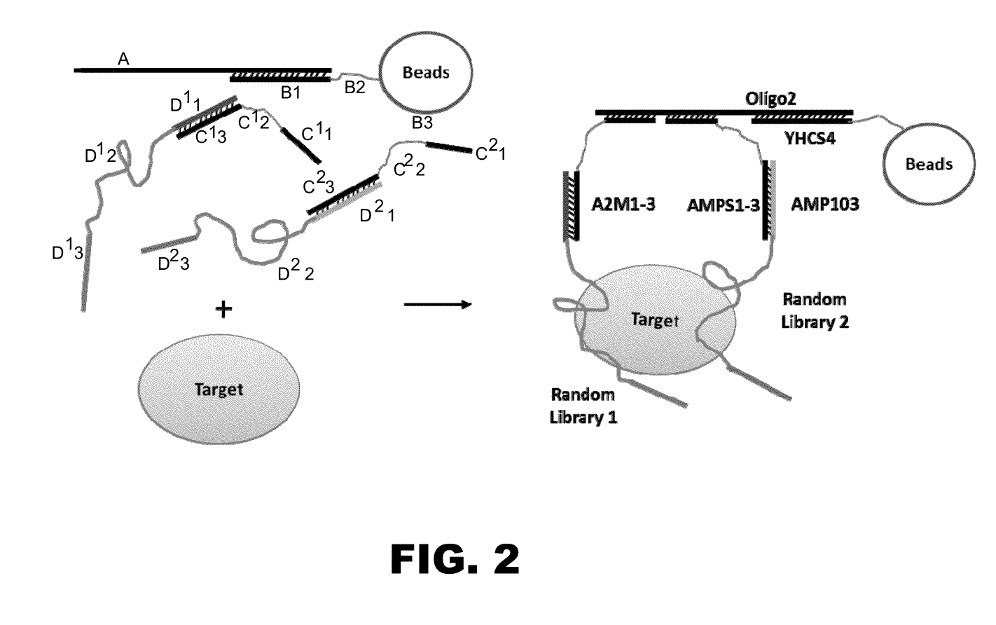

FIG. 2 depicts an illustration showing a composition for use in the simultaneous screening of a pair of aptamers against a target.

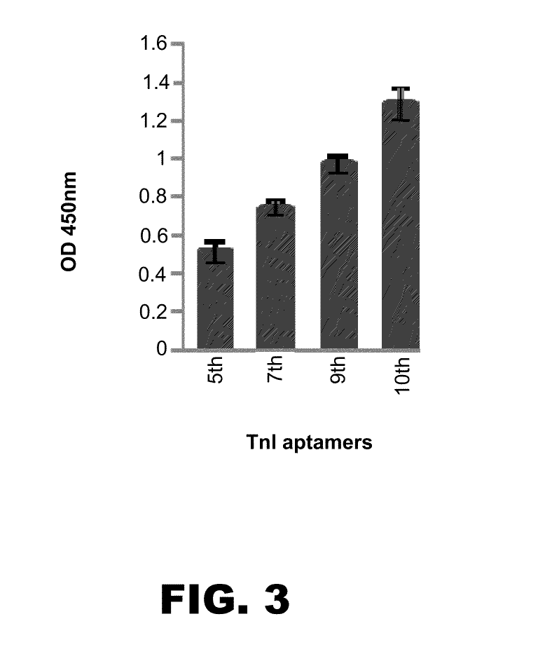

FIG. 3 depicts a graph showing the enrichment of the TnI specific aptamers.

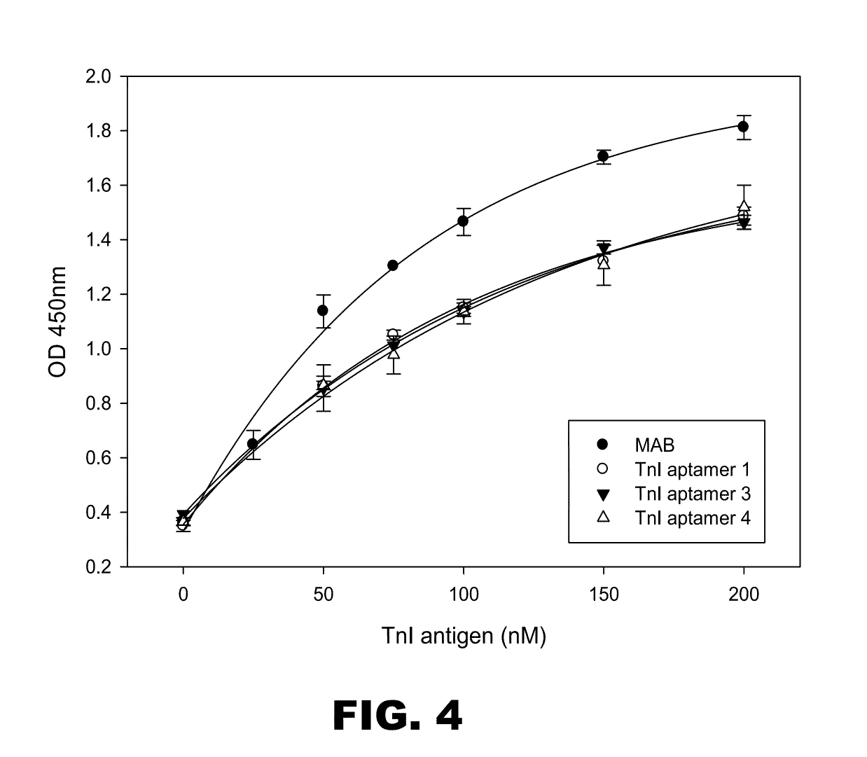

FIG. 4 depicts a graph showing the binding affinities of the different aptamers to TnI protein.

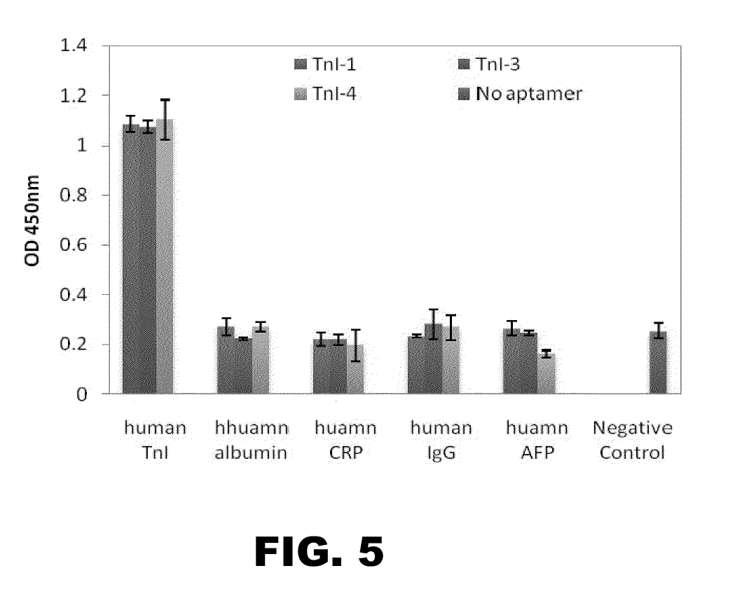

FIG. 5 depicts a graph showing the specificity of the TnI aptamers.

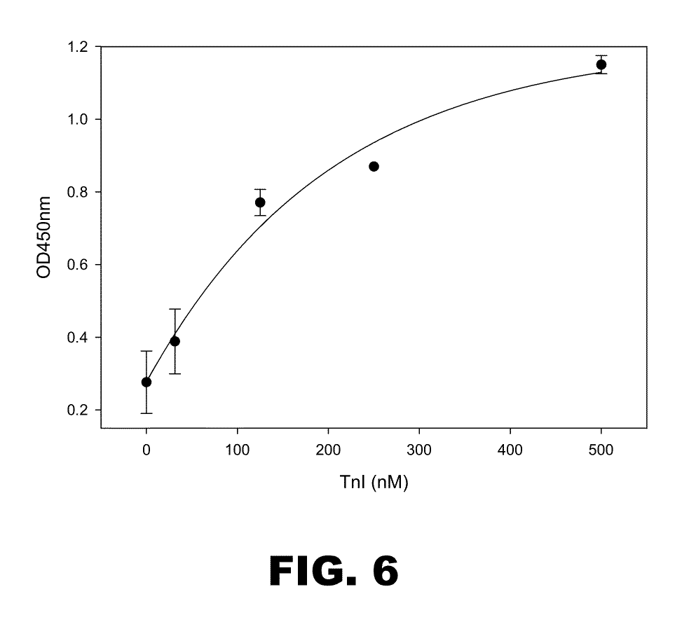

FIG. 6 depicts a graph showing the results of a sandwich ELISA assay for TnI using polyclonal antibody and TnI-1 aptamer.

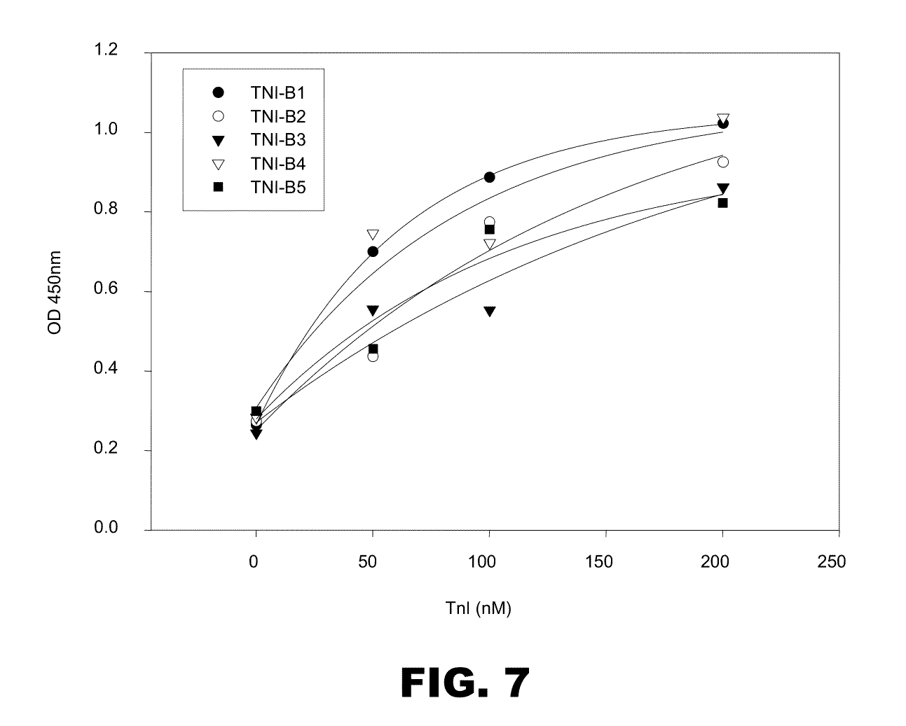

FIG. 7 depicts a graph showing the binding affinities of the aptamers to TnI protein.

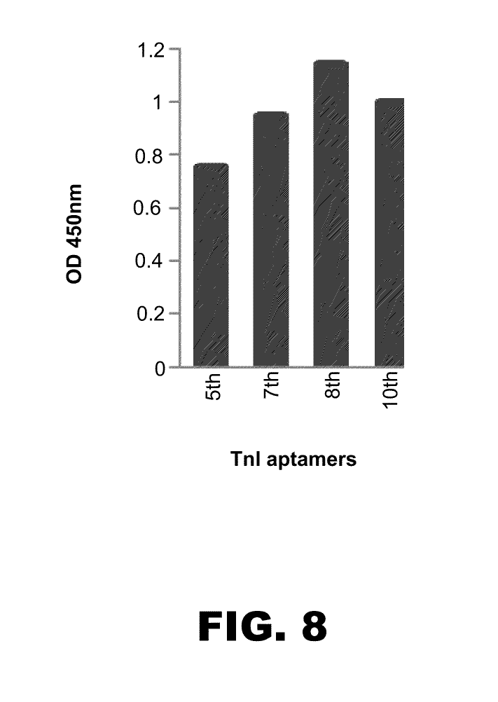

FIG. 8 depicts a graph showing the enrichment of the TnI specific aptamers.

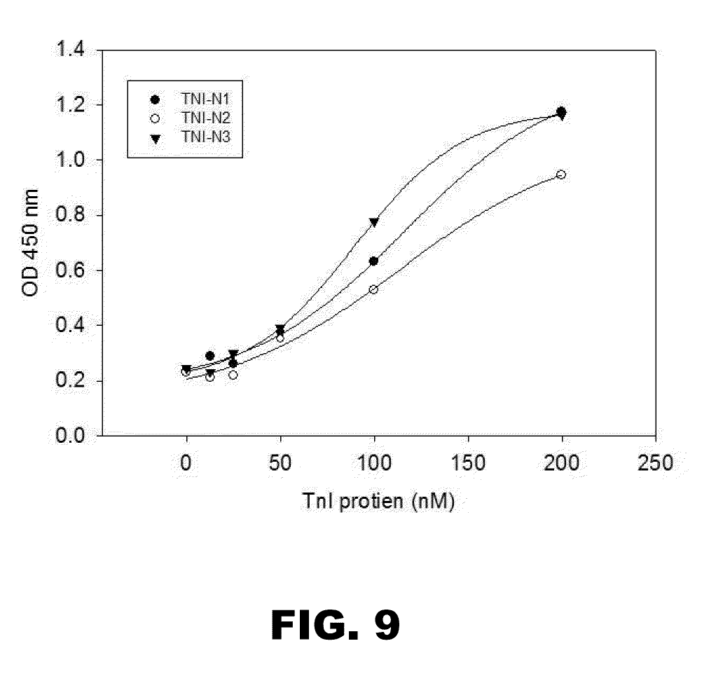

FIG. 9 depicts a graph showing the binding affinities of the aptamers to TnI protein.

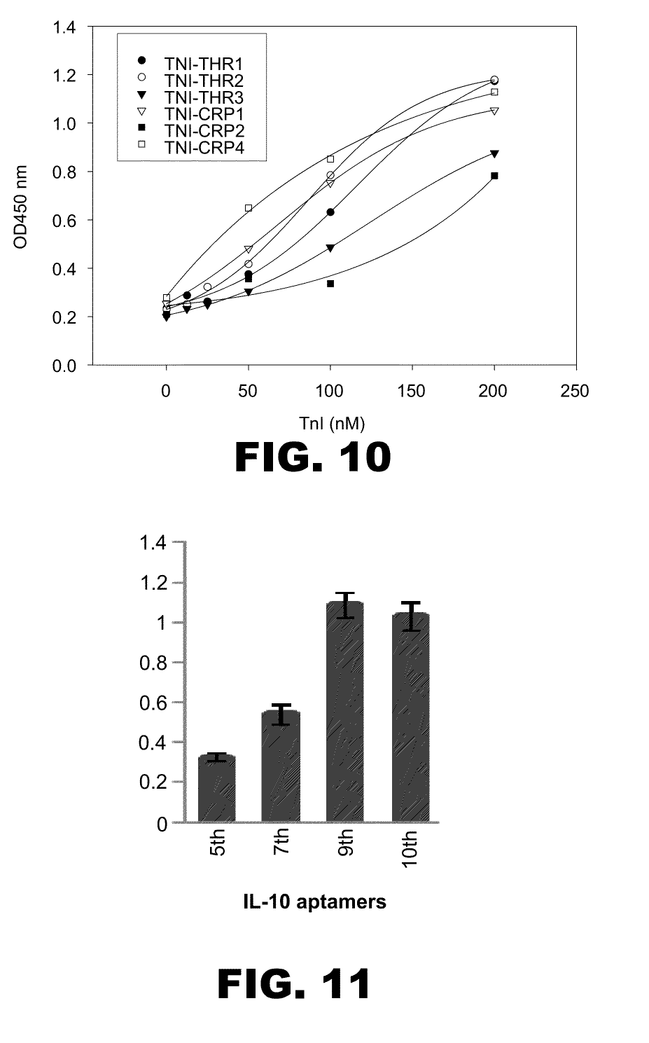

FIG. 10 depicts a graph showing the binding affinities of the aptamers to TnI protein.

FIG. 11 depicts a graph showing the enrichment of the IL-10 specific aptamers.

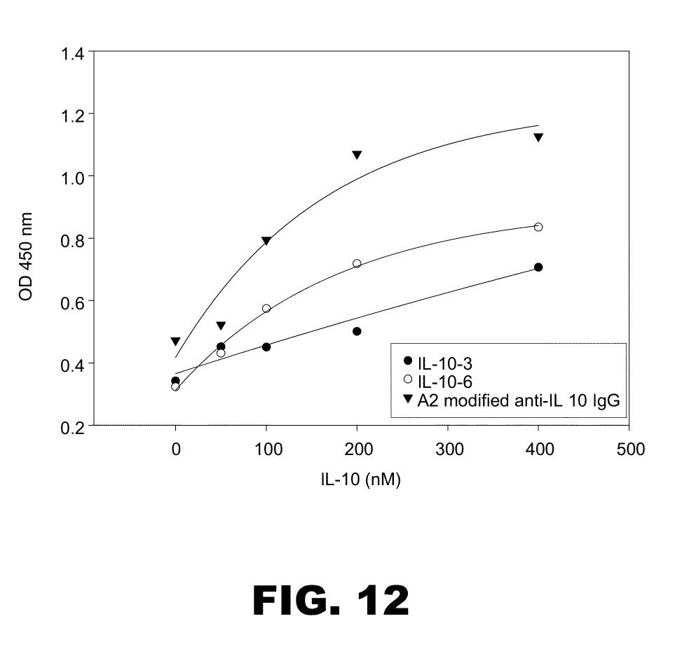

FIG. 12 depicts a graph showing the binding affinities of the aptamers and antibody to IL-10 protein.

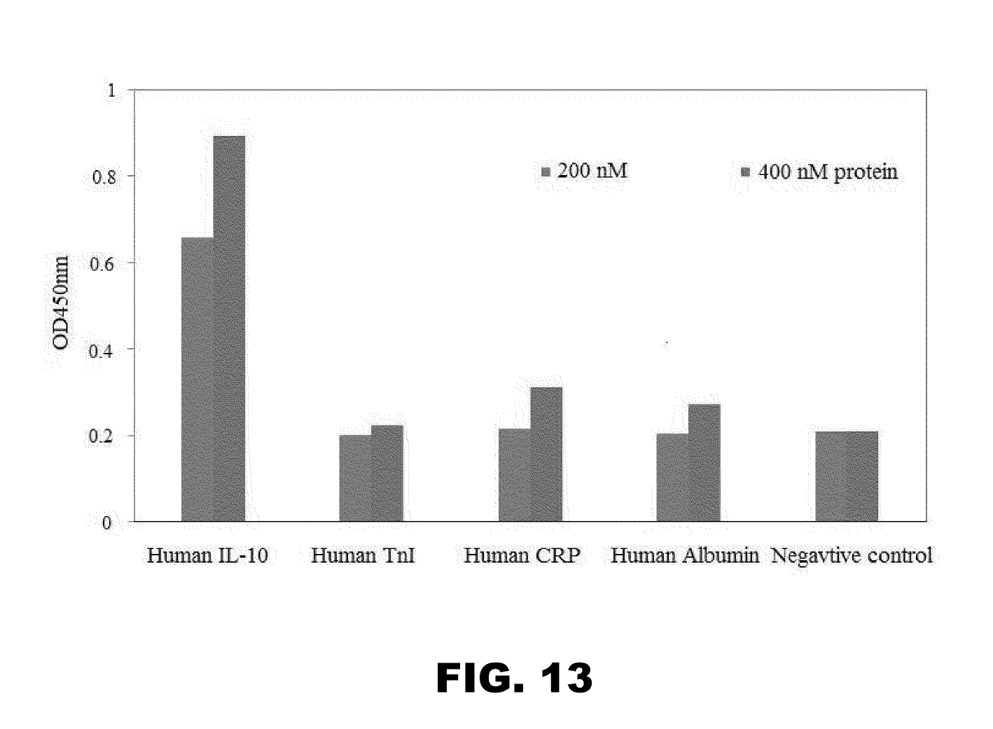

FIG. 13 depicts a graph showing the specificity of the IL-10 aptamers.

### DETAILED DESCRIPTION OF THE INVENTION

The present invention provides compositions and methods for selecting one or more aptamers that bind to a particular target or targets. Advantageously, the present compositions and methods provide an efficient and effective means to select aptamers.

### I. Compositions

One aspect of the present invention encompasses compositions for the selection of aptamers. In one embodiment, the present invention encompasses compositions for the selection of one or more aptamers in the presence of a known epitope binding agent. In another embodiment, the present invention encompasses compositions for the simultaneous selection of two or more aptamers without the presence of a known epitope binding agent.

In each embodiment, aptamers are selected for binding to a particular target. As used herein, “target” refers to one or more biomolecules, such as a protein, lipid, carbohydrate, a combination thereof, or a complex thereof.

Generally speaking, a composition of the invention minimally comprises three constructs. In one embodiment, a composition comprises a bridge construct (H1), at least one aptamer construct (I1-I2-I3-I4), and an epitope binding agent construct (J1-J2-J3). In another embodiment, a composition comprises a bridge construct (H1), and at least two aptamer constructs (I1-I2-I3-I4). Each construct is described in more detail below.

### (a) Bridge Construct

In one embodiment, a bridge construct comprises the construct H1. In another embodiment, a bridge construct comprises H1-H2-H3. H1 is a single-stranded nucleic acid, H2 is a linker that joins H1 and H3, and H3 is a solid support. Each of H1, H2, and H3 are described in more detail below.

H1 is a single-stranded nucleic acid that comprises a sequence complementary to each I1 (of a aptamer construct) present in a composition, and, if present, a sequence complementary to J1 (of an epitope binding agent construct). The orientation of the complementary sequences can and will vary. The complementary sequences may be adjacent, or may be separated. For instance, in a composition comprising an aptamer construct and an epitope binding agent construct, H1 comprises a sequence complementary to I1 and a sequence complementary to J1. The sequence complementary to I1 may be located 3′ to the sequence complementary to J1. Alternatively, the sequence complementary to J1 may be located 3′ of the sequence complementary to I1. The sequence complementary to I1 may be adjacent to the sequence complementary to J1, or there may be nucleic acid between them. In another example, in a composition comprising two aptamer constructs (I¹1-I¹2-I¹3-I¹4 and I²1-I²2-I²3-I²4) H1 comprises a sequence complementary to I¹1 and a sequence complementary to I²1. The sequence complementary to I¹1 may be located 3′ to the sequence complementary to I²1, or alternatively, the sequence complementary to I²1 may be located 3′ to the sequence complementary to I¹1. The sequence complementary to I¹1 may be adjacent to the sequence complementary to I²1, or there may be nucleic acid between them.

Typically, H1 should not be complementary to itself, i.e. H1 should not form a hairpin structure. H1 may comprise a natural nucleic acid (i.e. A, T, G, C, or U), or H1 may comprise a modified or synthetic nucleic acid. Modifications may occur at, but are not restricted to, the sugar 2′ position, the C-5 position of pyrimidines, and the 8-position of purines. Examples of suitable modified DNA or RNA bases may include 2′-fluoro nucleotides, 2′-amino nucleotides, 5′-aminoallyl-2′-fluoro nucleotides and phosphorothioate nucleotides (monothiophosphate and dithiophosphate). In some embodiments, H1 may comprise nucleotide mimics. Examples of nucleotide mimics may include locked nucleic acids (LNA), peptide nucleic acids (PNA), and phosphorodiamidate morpholine oligomers (PMO).

H2 is a linker that joins H1 and H3. H2 is optional, meaning that in some embodiments, H2 may be present, and in other embodiments, either H1 is joined directly to H3 without H2, or only H1 is present. Typically, H2 is flexible and may be comprised of natural nucleic acid, synthetic nucleic acid, other known linkers, such as bifunctional chemical linkers, or a combination thereof. For instance, H2 may be comprised of a natural or synthetic nucleic acid as described in relation to H1 above. In some embodiments, H2 is a bifunctional chemical linker or a polymer of a bifunctional chemical linker, such as a heterobifunctional linker (or a polymer thereof), a homobifunctional linker (or a polymer thereof), or a combination thereof. In one embodiment the bifunctional chemical linker is heterobifunctional. Suitable heterobifunctional chemical linkers may include sulfoSMCC (sulfosuccinimidyl-4-(N-maleimidomethyl)cyclohexane-1-carboxylate), and Ic-SPDP (N-succinimidyl-6-(3′-(2-pyridyldithio)-propionamido)-hexanoate). In another embodiment the bifunctional chemical linker is homobifunctional. Suitable homobifunctional linkers may include disuccinimidyl suberate, disuccinimidyl glutarate, and disuccinimidyl tartrate. Additional suitable linkers may include the phosphoramidate form of Spacer 18 comprised of polyethylene glycol. In an exemplary embodiment, H2 may be comprised of a bifunctional chemical linker and nucleic acid.

If H2 is a nucleic acid (natural or synthetic), H2 may be between about 0 and about 100 nucleotides in length. In one embodiment, H2 may be between about 10 to about 100 nucleotides in length. In another embodiment, H2 may be between about 10 to about 25 nucleotides in length. In yet another embodiment, H2 may be between about 25 to about 50 nucleotides in length. In a further embodiment, H2 may be between about 50 to about 75 nucleotides in length. In yet a further embodiment, H2 may be between about 75 to about 100 nucleotides in length.

In other embodiments, H2 may be between about 0 to about 500 angstroms in length. In some embodiments, H2 may be between about 20 to about 400 angstroms in length. In other embodiments, H2 may be between about 50 to about 250 angstroms in length.

H3 is a solid support. H3 is optional, meaning that in some embodiments, H3 may be present, and in other embodiments, only H1 is present (if there is no H3, there is typically no H2). Non-limiting examples of suitable solid supports may include microtitre plates, test tubes, beads, resins and other polymers, as well as other surfaces either known in the art or described herein. The solid support may be a material that may be modified to contain discrete individual sites appropriate for the attachment or association of the construct and is amenable to at least one detection method. Non-limiting examples of solid support materials may include glass, modified or functionalized glass, plastics (including acrylics, polystyrene and copolymers of styrene and other materials, polypropylene, polyethylene, polybutylene, polyurethanes, TeflonJ, etc.), nylon or nitrocellulose, polysaccharides, nylon, resins, silica or silica-based materials including silicon and modified silicon, carbon, metals, inorganic glasses and plastics. The size and shape of the solid support may also vary without departing from the scope of the invention. A solid support may be planar, a solid support may be a well, i.e. a 384 well plate, or alternatively, a solid support may be a bead or a slide. In an exemplary embodiment, H3 is a bead. In a further exemplary embodiment, H3 is a magnetic bead.

H3 may be attached to H2 (or H1 if H2 is not present) in a wide variety of ways, as will be appreciated by those in the art. H2, for example, may either be synthesized first, with subsequent attachment to H3, or may be directly synthesized on H3. H3 may be derivatized with chemical functional groups for subsequent attachment to H2 (or H1 if H2 is not present). For example, H3 may be derivatized with a chemical functional group including, but not limited to, amino groups, carboxyl groups, oxo groups or thiol groups. Using these functional groups, H1 may be attached using functional groups either directly or indirectly via H2. Alternatively, H2 may also be attached to the surface non-covalently. For example, a biotinylated H2 can be prepared, which may bind to H3 covalently coated with streptavidin, resulting in attachment. Alternatively, H2 may be synthesized on the surface using techniques such as photopolymerization and photolithography. In another alternative, H2 may be attached to H3 via a bifunctional chemical linker, and be attached to H1 via hybridization between complementary nucleic acids.

Additional methods of attaching H2 (H1) to H3 and methods of synthesizing nucleic acids on surfaces are well known in the art, i.e. VLSIPS technology from Affymetrix (e.g., see U.S. Pat. No. 6,566,495, and Rockett and Dix, “DNA arrays: technology, options and toxicological applications,” Xenobiotica 30(2):155-177, all of which are hereby incorporated by reference in their entirety).

In an exemplary embodiment, a bridge construct may be comprised of A and B1-B2-B3, such that A is similar to H1, B1 and B2 are similar to H2, and B3 corresponds to H3. In this regard, A is a single-stranded nucleic acid defined the same as H1 except A is not joined directly to a solid support. Rather, A hybridizes with B1. B1, in turn, is either joined with B3 (defined the same as H3) via B2 (defined the same as H2), or B1 is joined directly to B3, as detailed above with respect to H1 and H3.

### (b) Aptamer Construct

A aptamer construct of the invention usually comprises the construct I1-I2-I3-I4. I1 is a single-stranded nucleic acid that binds to a complementary region on H1.

I1 generally has a length such that the free energy of association between I1 and H1 is from about −5 to about −12 kcal/mole at a temperature from about 21° C. to about 40° C. and at a salt concentration from about 1 mM to about 100 mM. In other embodiments, the free energy of association between I1 and H1 is from about −5 kcal/mole, about −6 kcal/mole, about −7 kcal/mole, about −8 kcal/mole, about −9 kcal/mole, about −10 kcal/mole, about −I1 kcal/mole, or greater than about −12 kcal/mole at a temperature from about 21° C. to about 40° C. and at a salt concentration from about 1 mM to about 100 mM.

In some embodiments, I1 may range from about 4 to about 20 nucleotides in length. In other embodiments, I1 may be about 4, about 5, about 6, about 7, about 8, about 9, about 10, about 11, about 12, about 13, about 14, about 15, about 16, about 17, about 18, about 19, or greater than about 10 nucleotides in length.

I2 is a linker that joins I1 to I3. I2 is optional, meaning that in some embodiments, I2 may be present, and in other embodiments, I1 is joined directly to I3 without I2. I2 is defined the same as H2. In this regard, I2 is typically flexible and may be comprised of natural nucleic acid, synthetic nucleic acid, other known linkers, such as bifunctional chemical linkers, or a combination thereof as described for H2.

I3 is a potential aptamer sequence. As used herein, a “potential aptamer sequence” refers to a single stranded nucleic acid sequence that was not previously known to have specificity for a particular target. In some embodiments, I3 is a random sequence. In other embodiments, I3 is derived from a library of synthesized sequences. The length of I3 can and will vary, but generally speaking I3 is between about 10 nucleotides and 90 nucleotides long. In one embodiment, I3 is about 10, 15, 20, 25, 30, 35, 40, 45, 50, 55, 60, 65, 70, 75, 80, 85, or 90 nucleotides long. In another embodiment, I3 is about 25, 26, 27, 28, 29, 30, 31, 32, 33, 34, 35, 36, 37, 38, 39, 40, 41, 42, 43, 44, or 45 nucleotides long. I3 may be comprised of natural or synthetic nucleic acid, as defined for H1.

I4 is a single stranded nucleotide sequence with a known sequence that may be used as a primer sequence for a PCR reaction. As used herein, “primer sequence” refers to a sequence capable of hybridizing to a primer such that the primer initiates a polymerase chain reaction. I4 may vary in length, but generally speaking, will be between 10 and 50 nucleotides in long. In some embodiments, I4 may be about 10, 15, 20, 25, 30, 35, 40, 45, or 50 nucleotides long.

In an exemplary embodiment, an aptamer construct comprises C1-C2-C3 and D1-D2-D3, such that C1 corresponds to I1; C2, C3, and D1 correspond to I2; D2 corresponds to I3; and D3 corresponds to I4. Specifically, C3 is a single stranded nucleic acid that hybridizes to D1. Conversely, D1 is a single-stranded nucleic acid that hybridizes to C3. C3 may be joined with C1 (defined the same as I1) via C2 (defined the same as I2), or C3 is joined directly to C1 (e.g. C2 is not present).

In certain embodiments where it is desirable to select more than one aptamer at a time, a composition of the invention may comprise more than one aptamer construct. For example, a composition may comprise I¹1-I¹2-I¹3-I¹4 and I²1-I²2-I²3-I²4, where I¹1 and I²1 each bind to distinct regions on H1, but are not complementary to each other; I¹3 and I²3 are different potential aptamer sequences, and I¹4 and I²4 are each known primer sequences. In one embodiment, a composition may comprise 2, 3, 4, 5, 6, or more than 6 aptamer constructs. In an exemplary embodiment, a composition may comprise 2, 3, or 4 aptamer constructs.

### (c) Epitope Binding Agent Construct

An epitope binding agent construct of the invention usually comprises the construct J1-J2-J3. J1 is a single-stranded nucleic acid that binds to a complementary region on H1. Importantly, I1 and J1 are not complementary to each other, and I1 and J1 each bind to distinct regions on H1.

J1 generally has a length such that the free energy of association between J1 and H1 is from about −5 to about −12 kcal/mole at a temperature from about 21° C. to about 40° C. and at a salt concentration from about 1 mM to about 100 mM. In other embodiments, the free energy of association between I1 and H1 is from about −5 kcal/mole, about −6 kcal/mole, about −7 kcal/mole, about −8 kcal/mole, about −9 kcal/mole, about −10 kcal/mole, about −I1 kcal/mole, or greater than about −12 kcal/mole at a temperature from about 21° C. to about 40° C. and at a salt concentration from about 1 mM to about 100 mM.

In some embodiments, J1 may range from about 4 to about 20 nucleotides in length. In other embodiments, J1 may be about 4, about 5, about 6, about 7, about 8, about 9, about 10, about 11, about 12, about 13, about 14, about 15, about 16, about 17, about 18, about 19, or greater than about 10 nucleotides in length.

J2 is a linker that joins J1 to J3. J2 is optional, meaning that in some embodiments, J2 may be present, and in other embodiments, J1 is joined directly to J3 without J2. J2 is defined the same as H2. In this regard, J2 is typically flexible and may be comprised of natural nucleic acid, synthetic nucleic acid, other known linkers, such as bifunctional chemical linkers, or a combination thereof as described for J2.

J3 is an epitope binding agent that binds to a target. Non-limiting examples of suitable epitope binding agents, depending upon the target molecule, may include agents selected from the group consisting of an aptamer, an antibody, an antibody fragment, a double-stranded DNA sequence, modified nucleic acids, nucleic acid mimics, a ligand, a ligand fragment, a receptor, a receptor fragment, a polypeptide, a peptide, a coenzyme, a coregulator, an allosteric molecule, and an ion. In an exemplary embodiment, J3 is an aptamer having a sequence ranging in length from about 20 to about 110 bases. In another embodiment, J3 is an antibody selected from the group consisting of polyclonal antibodies, ascites, Fab fragments, Fab′ fragments, monoclonal antibodies, humanized antibodies, chimeric antibodies, and single-chain antibodies. In yet another embodiment, J3 is a peptide.

In an exemplary embodiment, an epitope binding agent construct comprises E1-E2-E3 and F1-F2-F3, such that E1 corresponds to J1; E2, E3, F1, and F2 correspond to J2; and F3 corresponds to J3. Specifically, E3 is a single stranded nucleic acid that hybridizes to F1. Conversely, F1 is a single-stranded nucleic acid that hybridizes to E3. E3 may be joined with E1 (defined the same as J1) via E2 (defined the same as J2), or E3 may be joined directly to E1 (e.g. E2 is not present). Similarly, F1 may be joined with F3 (defined the same as J3) via F2 (defined the same as J2), or F1 may be joined directly to F3 (e.g. F2 is not present).

### (d) Stable Complex with Target

A composition of the invention is designed to form a stable complex with a target. A stable complex of the invention requires at least three constructs, which constitutes at least four binding events: 1) aptamer construct to target, 2) aptamer construct to bridge construct, and either 3) second aptamer construct to target and 4) second aptamer construct to bridge construct or 3) epitope binding agent construct to target and 4) epitope binding agent construct to bridge construct.

When at least one aptamer construct is used in conjunction with at least one epitope binding agent construct, in some embodiments, I3 and J3 bind to different sites on the same target biomolecule. In this regard, J3 binds to a site on a target, and the potential aptamer (i.e. I3) is selected to bind to a site on the same target that is distinct from the J3 binding site. In other embodiments, I3 may bind to a first biomolecule, J3 may bind to a second biomolecule, and the first and second biomolecules may bind to each other to form a complex. In this regard, a stable complex would require five binding events: the first biomolecule to the second biomolecule, I3 to the first biomolecule, J3 to the second biomolecule, I1 to H1, and J1 to H1. In another embodiment, I3 may bind to a first biomolecule, J3 may bind to a second biomolecule, and the first and second biomolecules may each bind to a third biomolecule to form a complex. In this regard, a stable complex would require six binding events: the first biomolecule to the third biomolecule, the second biomolecule to the third biomolecule, I3 to the first biomolecule, J3 to the second biomolecule, I1 to H1, and J1 to H1. Other embodiments are possible, for instance, selecting an aptamer to a particular conformation of a target. This can be performed by incubating a composition of the invention with the target under conditions that favor a particular conformation of the target (e.g. in the presence of a particular metal, binding partner, salt concentration, etc.).

When at least two aptamer constructs are used without an epitope binding agent construct, the same target possibilities exist, including, in some embodiments, I¹3 and I²3 bind to different sites on the same target biomolecule. In this regard, I¹3 binds to a site on a target and I²3 is selected to bind to a site on the same target that is distinct from the I¹3 binding site. In other embodiments, I¹3 may bind to a first biomolecule, I²3 may bind to a second biomolecule, and the first and second biomolecules may bind to each other to form a complex. In this regard, a stable complex would require five binding events: the first biomolecule to the second biomolecule, I¹3 to the first biomolecule, I²3 to the second biomolecule, I¹1 to H1, and I²1 to H1. In another embodiment, I¹3 may bind to a first biomolecule, I²3 may bind to a second biomolecule, and the first and second biomolecules may each bind to a third biomolecule to form a complex. In this regard, a stable complex would require six binding events: the first biomolecule to the third biomolecule, the second biomolecule to the third biomolecule, I¹3 to the first biomolecule, I²3 to the second biomolecule, I¹1 to H1, and I²1 to H1. Other embodiments are possible, for instance, selecting an aptamer to a particular conformation of a target. This can be performed by incubating a composition of the invention with the target under conditions that favor a particular conformation of the target (e.g. in the presence of a particular metal, binding partner, salt concentration, etc.).

A “stable complex” is one that may be separated from the reaction mixture. By way of non-limiting example, H3 may be separated from the mixture (as when H3 is a bead), the reaction mixture may be washed away from H3 (as when H3 is a surface), or when H3 is not present, the reaction mixture may be washed over a filter to separate the complex of H1, the other two constructs, and target from the rest of the mixture. Because a stable complex requires at least four interactions, a composition of the invention is designed to select potential aptamer sequences with both specificity and affinity for a target, such that the binding event between each I3 (or I3 and J3) and a target contributes to the formation of a stable complex.

### (e) Exemplary Embodiments

In an exemplary embodiment, a composition of the invention comprises a combination of a bridge construct, an aptamer construct and an epitope binding agent construct listed in Table A. In a particular exemplary embodiment, a composition of the invention comprises combination number viii. of Table A, namely, A; B1-B2-B3; C1-C2-C3; D1-D2-D3; E1-E2-E3; and F1-F2-F3.

TABLE A
Possible composition combinations

| | | | Epitope binding |
|---|---|---|---|
| | Bridge construct | Aptamer construct | agent construct |
| i. | H1-H2-H3 | I1-I2-I3-I4 | J1-J2-J3 |
| ii. | H1-H2-H3 | I1-I2-I3-I4 | E1-E2-E3; F1-F2-F3 |
| iii. | H1-H2-H3 | C1-C2-C3; D1-D2-D3 | J1-J2-J3 |
| iv. | H1-H2-H3 | C1-C2-C3; D1-D2-D3 | E1-E2-E3; F1-F2-F3 |
| v. | A; B1-B2-B3 | I1-I2-I3-I4 | J1-J2-J3 |
| vi. | A; B1-B2-B3 | I1-I2-I3-I4 | E1-E2-E3; F1-F2-F3 |
| vii. | A; B1-B2-B3 | C1-C2-C3; D1-D2-D3 | J1-J2-J3 |
| viii. | A; B1-B2-B3 | C1-C2-C3; D1-D2-D3 | E1-E2-E3; F1-F2-F3 |

In other exemplary embodiments, a composition of the invention may comprise a combination of a bridge construct, and two aptamer constructs listed in Table B. In a particular exemplary embodiment, a composition of the invention comprises combination number viii. of Table B, namely, A; B1-B2-B3; D11-D12-D13; C21-C22-C23; and D²1-D²2-D²3.

TABLE B
Possible composition combinations

| | | First Aptamer | Second Aptamer |
|---|---|---|---|
| | Bridge construct | construct | construct |
| i. | H1-H2-H3 | I¹1-I¹2-I¹3-I¹4 | I²1-I²2-I²3-I²4 |
| ii. | H1-H2-H3 | I¹1-I¹2-I¹3-I¹4 | C²1-C²2-C²3; D²1-D²2- |
| | | | D²3 |
| iii. | H1-H2-H3 | C¹1-C¹2-C¹3; D¹1- | I²1-I²2-I²3-I²4 |
| | | D¹2-D¹3 | |
| iv. | H1-H2-H3 | C¹1-C¹2-C¹3; D¹1- | C²1-C²2-C²3; D²1-D²2- |
| | | D¹2-D¹3 | D²3 |
| v. | A; B1-B2-B3 | I¹1-I²-I¹3-I¹4 | I²1-I²2-I²3-I²4 |
| vi. | A; B1-B2-B3 | I¹1-I¹2-I¹3-I¹4 | C²1-C²2-C²3; D²1-D²2- |
| | | | D²3 |
| vii. | A; B1-B2-B3 | C¹1-C¹2-C¹3; D¹1- | I²1-I²2-I²3-I²4 |
| | | D¹2-D¹3 | |
| viii. | A; B1-B2-B3 | C¹1-C¹2-C¹3; D¹1- | C²1-C²2-C²3; D²1-D²2- |
| | | D¹2-D¹3 | D²3 |

### II. Methods

Another aspect of the present invention is a method of using a composition of the invention to select one or more aptamers. In one embodiment, a method of the invention comprises contacting a composition of the invention with a target, separating a stable complex from the reaction mixture, and determining the identity of the aptamer sequence(s) that recognize the target. Each of these steps is discussed in more detail below.

(a) Contacting a Composition of the Invention with a Target

A method of the invention initially comprises contacting a composition of the invention with a target. The composition typically comprises at least three constructs—a bridge construct, and either two or more aptamer constructs or an epitope binding agent construct and one or more aptamer constructs. As defined in Section I above, a target may comprise a biomolecule, a combination of more than one biomolecules, or a complex of more than one biomolecule. A composition of the invention is typically contacted by a target under conditions suitable for binding of the potential aptamer sequence(s) to the target to form a stable complex. As detailed in section I(e) above, a stable complex requires at least four binding events. Suitable reaction mixtures (e.g. buffers, stabilizers, etc) and suitable reaction conditions are known in the art, or may be readily experimentally determined. For specific examples, please see the Examples herein.

(b) Separating the Stable Complex from the Reaction Mixture

Once a composition of the invention has been contacted with a target, a method of the invention comprises separating one or more stable complexes from the reaction mixture comprising unbound aptamer constructs and/or other components. This may be accomplished using several different means known in the art. For instance, if the composition comprises a bridge construct comprising a bead, the solution may be centrifuged, pelleting the bead(s) and then washing them to remove unbound aptamer constructs and/or other components. Alternatively, if the beads are magnetic, a magnet may be used to separate the beads from the reaction mixture. If the composition comprises a bridge construct comprising a flat surface, such as a well or glass slide, then the stable complex may be isolated by washing the flat surface to remove unbound aptamer constructs and/or other components. In another alternative, if the bridge construct does not comprise a solid surface (i.e. H3), than a stable complex may be separated from the reaction mixture by filtering the mixture using nitrocellulose. Other methods of separation are known in the art and may be utilized by a skilled artisan.

### (c) Identifying the Aptamer Sequence

Once the stable complex is separated from the reaction mixture, then a PCR reaction may be used to amplify the sequence of the aptamer(s) that bound the target. For each aptamer construct, a primer that anneals to I4 may be used in conjunction with a primer that anneals to I2. For instance, in one embodiment, a primer anneals to a sequence in C3. One primer may be fluorescently labeled. Another primer may be labeled with biotin. In certain embodiments, a double-stranded PCR product may comprise both a fluorescently labeled primer and a biotin labeled primer.

After amplification, a PCR product may be separated on an agarose gel using electrophoresis. The band comprising the aptamer may be excised from the gel, and the DNA may be eluted and sequenced to determine the aptamer sequence that bound the target. For more detailed information, please see the Examples submitted herewith.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that may changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Example 1

### Screening of Aptamers Against a Target with the Assistance of an Existing Epitope Binding Agent

The materials and methods for creating a composition as illustrated in FIG. 1 and described in section I(a) above are as follows:

1. Modification of Antibody:

The oligonucleotide AMP103 is modified with SM PEG(12) linker (Pierce, Ill.) by mixing AMP103 and linker in 75 μl PBS at final concentration of 200 μM and 4 mM, respectively. The amino groups of antigen-specific antibody are partially converted to SH group by mixing 200 μg antibody with Traut's reagent (Pierce, Ill.) in 200 μl PBS and incubating at room temperature. The antibody is then mixed with AMP103-PEG(12) mentioned above and incubated at room temperature for 6 hours. The modified antibody is purified by gel filtration chromatography. The concentrations of AMP103 and antibody are determined by UV absorbance and BCA protein assay kit (Pierce, Ill.), respectively. The modified antibody is hybridized to AMPS1-3 at 1.8:1 (AMP103:AMPS1-3) molar ratio in TBS.

2. Immobilization of Oligo 2/YHCS4 on the Beads Containing Free Amino Groups:

The oligonucleotide YHCS4-SS is first reduced by incubating with 50 mM DTT at room temperature. The reduced oligo is desalted twice using G-25 microspin column (GE). 100 μl of amino beads (Solulink, CA) are conjugated to PEG12 linker by incubating with 1 mg SM PEG(12) linker in final 500 μl PBS buffer. The beads are washed with PBS and react with the reduced YHCS4 in PBS. The beads are washed MEDIee tims and incubated with excess amount of Oligo2 in TBS buffer with 200 mM NaCl and 1 mM MgCl₂. The beads are washed and stored in TBS buffer. The immobilization rate is determined via SYBR Green staining.

3a. Initial Round of SELEX Performed on Beads with the Assistance of Antibody:

The random DNA library MEDI-11 (containing 30 or 39 nucleotide-long sequences) and A2M1-4 in 500 μL SELEX buffer (50 mM Tris, pH 7.5, 100 mM NaCl, 5 mM KCl, 1 mM MgCl₂) are boiled for 1 minute in water bath and cooled down slowly to room temperature. After the beads are pulled down, the supernatant is collected, mixed with Oligo2/YHCS4 immobilized on beads, protein target, and antibody-AMP103/AMPS1-3/block LT. After incubation at 24° C. for 30 min, the beads are pulled down and the supernatant is removed. The beads are gently washed with 200 μL SELEX buffer, and the bound aptamers are recovered and separated from the beads by suspending the beads in ddH₂O. The suspension is incubated at 46° C. for 10 min, after which the beads are pulled down on magnetic set. The supernatant is concentrated to 40 μL and 20 μL of it is used as template for the PCR reaction. The PCR product is then separated from primers on 10% native PAGE. The product bands are cut and collected in an eppendorf tube. The DNA is recovered by soaking the smashed gel pieces in the elution buffer (100 mM Tris, pH 8.0, 0.5 M NaCl, 5 mM EDTA) for at least 2 hours at 60° C. The supernatant is collected and mixed with pre-equilibrated NanoLink™ streptavidin beads (10 mg/ml) (Solulink, Inc. CA). After incubation for 1.5 hour at room temperature, the beads are washed with the washing buffer (50 mM Tris-HCl, pH 8.0, 150 mM NaCl, 0.05% Tween 20). The fluorescent-labeled strand is then separated from the biotin-labeled strand by treating the beads with NaOH solution for 1 min at 24° C. The beads are pulled down using magnetic set and the supernatant is collected, quickly neutralized with HCl. The aptamer containing supernatant is desalted using a G-25 microspin column (pre-equilibrated twice with the SELEX buffer), and the concentration is determined based on comparison of the emission of FAM to the FAM labeled primer standards.

3b. Initial Round of SELEX Performed on Nitrocellulose Filter (NCF) with the Assistance of Antibody:

The random DNA library MEDI-11 (containing 30 or 39 nucleotide-long random sequences) and A2M1-4 in 500 μL SELEX buffer (50 mM Tris, pH 7.5, 100 mM NaCl, 5 mM KCl, 1 mM MgCl2) is boiled for 1 min in water bath and cooled down slowly to room temperature. To get rid of the aptamers binding to NCF filter, the mixture is pre-incubated with a NCF filter (that has been pre-equilibrated with 1×0.5 ml NaOH solution and 2×1 ml SELEX buffer) for 20 minutes at 24° C. The mixture is spinned through the NCF filter and the flow through are collected and mixed with the target protein, Oligo 2R/YHCS4-S, and AMPS1-3 of anti-target-AMP103/AMPS1-3/block LT. After incubation at room temperature for 30 min, the mixture is spinned through another NCF filter and the NCF filter is washed by 2×0.5 ml SELEX buffer. After washing, fresh urea buffer is added to the filter, incubated at 37° C. for 15 min, and the DNA-urea mixture is spinned down to a fresh microtube. The selected aptamer is recovered by ethanol precipitation and dissolved in dH₂O. The recovered DNA is used as template for PCR reaction using FAM labeled primer MEDIP1 and biotin labeled primer MEDIP2. The PCR product is separated from primers on 10% native PAGE and recovered according to the procedure described in Part 1, Step 3a.

4. Second and Later Round Performed on Beads:

A mixture of aptamer (from last round) and OligoA2-M1-3 in SELEX buffer is boiled for 1 min in water bath and cooled down slowly to room temperature. To get rid of the aptamers binding to Oligo2/YHCS4 immobilized beads, Oligo2/YHCS4 modified magnetic beads are added to the DNA mix and incubated at room temperature for 30 min. After incubation, the beads are pulled down using magnetic set and the supernatant is collected. Oligo2/YHCS4 modified magnetic beads, target, AMPS1-3 and anti-target-AMP103/AMPS1-3/block LT are added to the DNA mixture and incubated at room temperature for 30 min. After incubation, the supernatant is removed and the beads are gently washed twice for every round until the 4^{th} and three times for the rest rounds with 200 μl SELEX buffer. The bound aptamers are recovered and separated from the beads by suspending the beads in 50 μl of dH₂O and incubating at 46° C. for 10 min, followed by pulling down the beads using magnetic set. The supernatant is used as template for PCR reaction. The rest of the procedures are the same as initial round. The procedure 4 is repeated until aptamers with reasonable affinity are selected and confirmed by binding assay.

5. Determine Binding Affinity:

To determine the binding affinity, target protein is coated on 384-well ELISA plate overnight at 4° C. with concentrations ranging from 0 to 1 μM. Each well is blocked with 1% BSA 4 hours at room temperature. After washing twice with SELEX buffer, 30 μl of 50 nM aptamer/biotin-labeled primer in SELEX buffer with 0.1 mg/ml BSA (pre-hybridized by boiling 1 min and slowly cooling down to room temperature) is added to each well and incubated for 1 hr at room temperature. After washing twice with 100 μl SELEX buffer, 30 μl streptavidin-HRP (Pierce) 1:2000 diluted in SELEX buffer with 0.2 mg/ml BSA is added and incubated for 30 min. After washing three times with 100 μl SELEX buffer, 30 μl of TMB/H₂O₂ is added to each well. After the color is developed, 30 μl of 2 M H₂SO₄ is added to each well to stop the reaction. The OD 450 nm is recorded using a TECAN plate reader.

6. The Competition Assay by ELISA

To determine whether the aptamer compete with each other, target protein is coated on 384-well ELISA plate overnight at 4° C. with a concentration resulting in 80% saturation observed from above binding affinity assay. Each well is blocked with 1% BSA for 4 hr at 24° C. After washing MEDIee times with 100 μl SELEX buffer, 30 μl of 50 nM aptamer/biotin-labeled primer (prehybridized by boiling 1 min and slowly cooling down to room temperature), and increasing concentrations of competiting aptamer in SELEX buffer with 0.2 mg/ml BSA, are added to each well and incubated for 1 hr at room temperature. 30 μl streptavidin-HRP (Pierce) 1:2000 diluted in SELEX buffer with 0.2 mg/ml BSA is added and incubated for 30 min. After washing MEDIee times with 100 μl SELEX buffer, 30 μl of TMB/H₂O₂ is added to each well. After the color is developed, 30 μl of 2 M H₂SO₄ is added to each well to stop the reaction. The OD 450 nm is recorded using a TECAN plate reader.

7. Clone and Sequence the Aptamers:

After the reasonable binding affinity is achieved and confirmed by ELISA assay, the aptamer from the last round is PCR amplified using unlabeled corresponding primer pair. The PCR product is separated on 10% native acrylamide gel and the DNAs are stained with SYBR Green. The band containing the PCR product is cut and the DNAs are eluted. After ethanol precipitation, the DNAs are end converted and ligated into pETBlue-3 vector and transformed into NovaBlue competent cells. The competent cells are plated on LB agar plate containing ampicillin, X-gal and IPTG. 10 to 20 white colonies are picked up and checked by PCR using T7 promoter primer and reverse primer on the vector to ensure the insertion of the PCR products. Each clone is marked and grown in 4 ml LB medium containing ampicillin. The DNA is isolated using plasmid DNA isolation kit and sent to Retrogen for DNA sequencing. The identified aptamer sequences are synthesized from IDT and checked the affinity to target using ELISA assay as described above in step 5.

### Example 2

### Simultaneous Screening a Pair of Aptamers Against a Target

The materials and methods for creating a composition as illustrated in FIG. 2 and described in section I(b) above are as follows:

1. Initial Round of SELEX:

The random DNA library MEDI-11 and random DNA library CRP1-30 in total of SELEX buffer are boiled for 1 min and slowly cooled down to 24° C. The mixture is spinned through a NCF filter that is pre-equilibrated with 1×0.5 ml of NaOH solution and 2×1 ml SELEX buffer. The flow through is collected. The target protein is added to the flow through and well mixed. After incubation at room temperature for 30 min, the mixture is spinned through another nitrocellulose filter (pre-equilibrated) and followed by 2×1 ml SELEX buffer wash. After washing, fresh urea buffer is added to the filter, incubated at 37° C. for 15 min, and the DNA-urea mixture is spinned down to a fresh microtube. The selected aptamer is recovered by ethanol precipitation and dissolved in 20 μl dH₂O. The solution is each used as a template for PCR reactions with either primer pair FAM-MEDIP1/biotin-MEDIP2 or FAM-CRP1/biotin-CRP2. The rest of the procedures are the same as described for the initial round of “Screening of aptamers against a target with the assistance of an existing binder”.

2. Second and Later Rounds:

The aptamers of both libraries from last round are mixed together with oligo A2-M1-3 and oligo AMPS1-3 in SELEX buffer. The mixture is boiled for 1 min in water bath and cooled down slowly to room temperature. Oligo2/YHCS4 modified magnetic beads are added to the mixture. After incubation at room temperature for 30 min, the beads are pulled down using magnetic set and the supernatant is collected. Oligo2/YHCS4 modified magnetic beads and target are added to the DNA mixture. After incubation at room temperature for 30 min, the supernatant is removed and beads are washed with 200 μL of SELEX buffer. The aptamers are recovered and separated from the beads by suspending the beads in 60 μL of ddH₂O and incubating at 46° C. for 10 min, followed by pulling down the beads using the magnetic set. 20 μL of the supernatant is used as template for 250 μL PCR reaction of the MEDI11 library and the CRP1-30 library, respectively. The rest of the procedures are the same as the 1st round. The same procedure is repeated until aptamers with reasonable affinity are selected and confirmed by binding assay.

3: Evaluate Binding Affinity and Cloning of the Aptamers:

The binding affinity and the competition assay, and the cloning and sequencing procedures of the aptamer selected from the two libraries are the same as in “Screening of aptamers against a target with the assistance of an existing binder.”

### Example 3

### Screening of Aptamers Against Troponin I with Ab Assistance

Screening of aptamers against Troponin I with the assistance of antibodies (the initial round was performed on NCF without antibody) was performed using a 30 nt ssDNA library (MEDI-11-30) using the procedure described in Example 1. The initial round was performed using the procedure described in 3c. A monoclonal anti-hTnI antibody or a polyclonal anti-hTnI antibody for the C-terminal tail of hTnI protein (Biospacific) was used as bait in each round except the initial round. After the 10^{th} round, ELISA assay was performed to evaluate the binding of the aptamers to TnI protein. The ELISA results (FIG. 3) show the enrichment of the aptamers specific for TnI protein. The aptamers from the 10^{th} round were cloned and the DNA sequences were obtained (Table 1). Four aptamers have been identified, two of which have similar sequences with one nucleotide difference. The aptamers were synthesized and the bindings were evaluated using ELISA. All of them have very similar affinity to TnI protein.

TABLE 1
Sequences for Troponin I aptamers selected
with the assistance of antibodies (the initial
round was performed on NCF without antibody).

| | | SEQ |
|---|---|---|
| | | ID |
| Name | Nucleotide Sequence | NO: |
| TNI-1 | 5′CTGTCGTTAGTGAAGGTTGGCAACAACGACCGGGACTA | 1 |
| | ACGGCAGCAGCGACACCGGTAACGCCATATCACAGACG 3′ | |
| TNI-2 | 5′CTGTCGTTAGTGAAGGTTGGTAACAACGACCGGGACTA | 2 |
| | ACGGCAGCAGCGACACCGGTAACGCCATATCACAGACG 3′ | |
| TNI-3 | 5′CTGTCGTTAGTGAAGGTTGGCGGCGGACCGGAACGAG | 3 |
| | CGGCAGTCGCAGTCGACCCCAACGCCATATCACAGACG 3′ | |
| TNI-4 | 5′CTGTCGTTAGTGAAGGTTGCCAAGCGAACTGCGACAG | 4 |
| | GGGATCCAGGAGACGGCCCCAACGCCATATCACAGACG 3′ | |

To further characterize the selected aptamers, the affinities of the aptamers to TnI were compared with the affinity of anti-TnI monoclonal antibody and the affinities of aptamers were calculated based on the affinity of antibody (FIG. 4) (Table 2). The specificities of the aptamers were also evaluated using ELISA assay. The selected aptamers are specific for human troponin I protein and did not cross react with human albumin, human CRP, human IgG, and human alpha feto-protein (FIG. 5).

TABLE 2
The affinities of the Anti-TnI antibody and the aptamers.

| Binder | Kd (M) |
|---|---|
| Monoclonal anti-TnI | 5.5 × 10⁻⁸ |
| TNI-1 | 2.2 × 10⁻⁷ |
| TNI-3 | 2.2 × 10⁻⁷ |
| TNI-4 | 2.0 × 10⁻⁷ |

To explore the application of the selected aptamers, we used one of the aptamer, TnI-1, to pair up with anti-human TnI polyclonal antibody for sandwich ELISA. Anti-human TnI polyclonal antibody was coated on 384-well ELISA plate to capture the TnI protein from samples. TnI-1 was used as the detect reagent to bind to the TnI protein retained on the plate. Signal was developed based on the streptavidin-HRP (binds to biotinylated aptamer) catalyzed colorimetric reaction (FIG. 6). There was a TnI protein concentration dependent increase of the signal, indicating the aptamer TnI-1 could be paired up with the polyclonal anti-TnI antibody.

### Example 4

### Screening of Aptamers with Beads, Using Ab Assistance

Screening of aptamers against Troponin I with the assistance of antibodies (the initial round was performed on beads with antibody assistance) was performed using a 39 nt ssDNA library (MEDI-11-39) using the procedure described in Part 1. The initial round was performed using the procedure described in Step3a. A polyclonal anti-hTnI antibody for the C-terminal tail of hTnI protein (Biospacific) was used as bait in each round including the initial round. After the 10^{th} round, ELISA assay was performed to evaluate the binding of the aptamers to TnI protein. The aptamers from the 10^{th} round were cloned and the DNA sequences were obtained (Table 3). 5 aptamers have been identified, one of which (TNI-B1) has the exact same sequence as TNI-1 identified previously when the initial round was done on NCF without the assistance of antibody. The aptamers were synthesized and the bindings were evaluated using ELISA (FIG. 7).

TABLE 3
Aptamer sequences for TnI done on beads with antibody
assistance.

| | | SEQ |
|---|---|---|
| Name | Nucleotide Sequence | ID NO: |
| TNI-B1 | 5′CTGTCGTTAGTGAAGGTTGGCAACAACGACCGGGACTAACGGCA | 5 |
| | GCAGCGACACCGGTAACGCCATATCACAGACG 3′ | |
| TNI-B2 | 5′CTGTCGTTAGTGAAGGTTGCAGGCACGCTGCGATATGTCCAGTTG | 6 |
| | ACCCGGTTTGCCAACGCCATATCACAGACG 3′ | |
| TNI-B3 | 5′CTGTCGTTAGTGAAGGTTGGCGGCGGACCGGAACTAGCGGCAGT | 7 |
| | CACAGTCGACCCCAACGCCATATCACAGACG 3′ | |
| TNI-B4 | 5′CTGTCGTTAGTGAAGGTTGCCCCGGGAACCGAAACTTGCGACCT | 8 |
| | GTCGTCACCTTGTAACGCCATATCACAGACG 3′ | |
| TNI-B5 | 5′CTGTCGTTAGTGAAGGTTGGCGGCGGACCGGAACGAACGGCAGC | 9 |
| | AGCGACACCGGTAACGCCATATCACAGACG 3′ | |

### Example 5

### Screening of Aptamers Against Troponin I

Screening of aptamers against Troponin I with the assistance of antibodies (the initial round was done on NCF with antibody) was performed using a 39 nt ssDNA library (MEDI-11-39) following the procedure described in Example 1. The initial round was performed using the procedure described in Step3b. A monoclonal anti-human TnI antibody (Biospacific) was used as bait in each round including the initial round. After the 10^{th} round, ELISA assay was performed to evaluate the binding of the aptamers to TnI protein. The ELISA results (FIG. 8) show the enrichment of the aptamers specific for TnI protein. The aptamers from the 10^{th} round were cloned and the DNA sequences were obtained (Table 4). Three aptamers have been identified, one of which (TNI-N1) has the exact same sequence as TNI-1 identified previously. The aptamers were synthesized and the bindings were evaluated using ELISA (FIG. 9).

Simultaneous screening a pair of aptamers against Troponin I was performed using a 39 nt ssDNA library MEDI-11-39, and a 30 nt ssDNA library CRP1-30, following the procedure described in Part 2. After the 10th round, ELISA assay was performed to evaluate the binding of the aptamers to TnI protein. The aptamers from the 10th round were cloned and the DNA sequences were obtained (Table 4). Three aptamers have been identified from MEDI library, one of which (TNI-MEDI1) has the exact same sequence as TNI-1 identified previously. Four aptamers have been identified from CRP library (Table 5). The aptamers were synthesized and the bindings were evaluated using ELISA (FIG. 10).

TABLE 4
Aptamer sequences for TnI done on NCF with antibody assistance.

| Name | Nucleotide Sequence | SEQ ID NO: |
|---|---|---|
| TNI-N1 | 5′CTGTCGTTAGTGAAGGTTGGCAACAACGACCGGGACTAAC | 10 |
| | GGCAGCAGCGACACCGGTAACGCCATATCACAGACG 3′ | |
| TNI-N2 | 5′CTGTCGTTAGTGAAGGTTGCAGGCACGCTGCGATATGTCC | 11 |
| | AGTTGACCCGGTTTGCCAACGCCATATCACAGACG 3′ | |
| TNI-N3 | 5′CTGTCGTTAGTGAAGGTTGGCGCAGGAACCTGAACGTTCG | 12 |
| | ACGGGGTCGGCACCTGTAACGCCATATCACAGACG 3′ | |

TABLE 5
Aptamer sequences identified for simultaneous SELEX.

| | | |
|---|---|---|
| Name | Nucleotide Sequence | SEQ ID NO: |

Aptamers from MEDI library

| | | |
|---|---|---|
| TNI-MEDI1 | 5'CTGTCGTTAGTGAAGGTTGGCAACAACGACCGGGACTAA | 13 |
| | CGGCAGCAGCGACACCGGTAACGCCATATCACAGACG 3' | |
| TNI-MEDI2 | 5'CTGTCGTTAGTGAAGGTTGGCGCAGGAACCTGAACGTTC | 14 |
| | GACGGGGTCGGCACCTGTAACGCCATATCACAGACG 3' | |
| TNI-MEDI3 | 5'CTGTCGTTAGTGAAGGTTGCCGCCCAACGCCATATCACA | 15 |
| | GACGGCCGCCCAACGCCATANCACAGACG 3' | |

Aptamers from CRP library

| | | |
|---|---|---|
| TNI-CRP1 | 5′TAGGTGCTCGACGCTGACCCAGACCCACCAATATATGCC | 16 |
| | CCGCGGTGTACTCAGTCGCAGGTCATG 3′ | |
| TNI-CRP2 | 5′TAGGTGCTCGACGCTGACCCAGGACGGCACCTTAACCG | 17 |
| | CGCGTGGGCTACTCAGTCGCAGGTCATG 3′ | |
| TNI-CRP3 | 5′TAGGTGCTCGACGCTGACCCAGGGGACACCTATCAATCG | 18 |
| | TCGTGCGGTACTCAGTCGCAGGTCATG 3′ | |
| TNI-CRP4 | 5′TAGGTGCTCGACGCTGACGGGCGACACCGAGGGGGCTG | 19 |
| | GGGCGCGGGTACTCAGTCGCAGGTCATG 3′ | |

### Example 6

### Screening of Aptamers Against IL-10

Screening of aptamers against IL-10 with the assistance of antibodies (the initial round was performed on NCF without antibody) was performed using a 30 nt ssDNA library (CRP1-30) using the procedure described in Example 1. The initial round was performed using the procedure described in Step3c. A monoclonal anti-human IL-10 antibody was used as bait in each round except the initial round. After the 10^{th} round, ELISA assay was performed to evaluate the binding of the aptamers to TnI protein. The ELISA results (FIG. 11) show the enrichment of the aptamers specific for IL-10 protein. The aptamers from the 10^{th} round were cloned and the DNA sequences were obtained (Table 5). 6 aptamers have been identified, two of which (IL-10-3 and IL-10-6) show potential binding to IL-10 protein (FIG. 12, Table 6). The specificities of the aptamers were evaluated using ELISA assay. The selected aptamers are specific for human IL-10 protein and did not cross react with human Troponin I, human albumin, human CRP (FIG. 13).

TABLE 6
Aptamer sequences identified for IL-10 protein.

| | | SEQ |
|---|---|---|
| | | ID |
| Name | Nucleotide Sequence | NO: |
| IL-10-1 | 5′TAGGTGCTCGACGCTGACCCAGGGCTACACCTT | 20 |
| | ATCCATTCGCGCGGTACTCAGTCGCAGGTCATG 3′ | |
| IL-10-2 | 5′TAGGTGCTCGACGCTGACCCAATCCTGACACAC | 21 |
| | CGTATTAAGCCGCGTACTCAGTCGCAGGTCATG 3′ | |
| IL-10-3 | 5′TAGGTGCTCGACGCTGACCCACGAAAGACACCG | 22 |
| | CAGTCCCTCCACGCTACTCAGTCGCAGGTCATG 3′ | |
| IL-10-4 | 5′TAGGTGCTCGACGCTGACCCCCCAGTCACACCT | 23 |
| | TATGACCGTCCCGCCACTCAGTCGCAGGTCATG 3′ | |
| IL-10-5 | 5′TAGGTGCTCGACGCTGACCCAGGCACACCTATC | 24 |
| | CAACTGTCACCGGCCACTCAGTCGCAGGTCATG 3′ | |
| IL-10-6 | 5′TAGGTGCTCGACGCTGACCGGGCATTGACACCT | 25 |
| | TATCGTGGGGTGGGGACTCAGTCGCAGGTCATG 3′ | |

TABLE 7
The affinities of the anti-IL-10 antibody and the aptamers.

| Binder | Kd (M) |
|---|---|
| A2 modified anti-IL-10 IgG | 1 × 10⁻⁷ |
| IL-10-3 | 2 × 10⁻⁶ |
| IL-10-6 | 1 × 10⁻⁶ |

TABLE 8
Oligonucleotide sequences used in this project in addition to the
aptamers.

| | | SEQ |
|---|---|---|
| Name | Sequence | ID NO: |
| Oligo 2 | 5′ A TAA GGT GTC GAC TGG ATT AG/isp18/ | 26 |
| | TAGGTGCTGCACGCTGACAAA3 | |
| YHCS4 | 5′ /5ThioMC6-D/ TTT TTT TTT TTT TTT GTC AGC | 27 |
| | GTG CAG CAC CTA 3′ | |
| YHCS4-bio | 5′ /5Biosg//iSp18/ TTT TTT TTT TTT TTT GTC | 28 |
| | AGC GTG CAG CAC CTA 3′ | |
| AMPS1-3 | 5′ GTC AGC GTC GAG CAC CTA /iSp18/ TTT TGT | 29 |
| | TTC CAG TC 3′ | |
| A2M1-3 | 5′ GAC ACC TAT GTT TT /iSp18/5 CGT CTG TGA | 30 |
| | TAT GGC GTT 3′ | |
| AMP103 | 5′ TAG GTG CTC GAC GCT GAC /3AmMO/ 3′ | 31 |
| BlockLT | 5′ GTC AGC GTC GAG CAC CTA 3′ | 32 |
| Library Medi11 | 5′ CTG TCG TTA GTG AAG GTT (N)_{30 or 39} AAC GCC | 33 |
| | ATA TCA CAG ACG 3′ | |
| Primer Medi12F | 5′ /56-FAM/ CTG TCG TTA GTG AAG GTT 3′ | 34 |
| Primer Medi13B | 5′ /5Biosg/ CGT CTG TGA TAT GGC GTT 3′ | 35 |
| Library CRP1-30 | 5′ TAG GTG CTC GAC GCT GAC (N)₃₀ | 36 |
| | ACTCAGTCGCAGGTCATG 3′ | |
| Primer CRP 1 | 5′ FAM TAG GTG CTC GAC GCT GAC 3′ | 37 |
| Primer CRP 2 | 5′ Biotin CAT GAC CTG CGA CTG AGT 3′ | 38 |
| THR-bridge | 5′ AAAG GTG TCG ACT CGT TTT CTG GAG CGA | 39 |
| | CTG GAT TAGTTTTAGGTGCTGCACGCTGAC 3′ | |
| A2M1 extend | ACG AGT CGA CAC CTT TGT TTT (pacer 18) | 40 |
| | CGTCTGTGATATGGCGTT 3′ | |
| AMPS extend | 5′ AACCTTCACTAACGACAG (spacer 18) TTT TGT | 41 |
| | ATC CAG TCG CTC CAG 3′ | |
| AMPS extend-2 | 5′ ATC CAG TCG CTC CAG TGT TTT (pacer 18) | 42 |
| | CGTCTGTGATATGGCGTT 3′ | |

1-12. (canceled)

13. A composition, the composition comprising three components: a bridge construct, and two aptamer constructs, wherein
a) the bridge construct comprises
H1-H2-H3;
b) the first aptamer construct comprises
I¹1-I¹2-I¹3-I¹4;
c) the second aptamer comprises
I²1-I²2-I²3-I²4;
wherein
H1 is a single-stranded nucleic acid comprising a binding site for I¹1 and I²1,
H2 is a linker that joins H1 and H3,
H3 is a solid support,
I¹1 is a single-stranded nucleic acid that binds to a complementary region on H1, such that when I¹3 and I²3 bind to a target molecule, I¹1 stably binds to H1, but in the absence of a target molecule, I¹1 does not stably bind to H1,
I¹2 is a linker that joins I¹1 to I¹3,
I¹3 is a potential aptamer sequence that binds a target,
I¹4 is a primer sequence,
I²1 is a single-stranded nucleic acid that binds to a complementary region on H1, such that when I²3 and I²3 bind to a target molecule, I²1 stably binds to H1, but in the absence of a target molecule, I²1 does not stably bind to H1,
I²2 is a linker that joins I²1 to I²3,
I²3 is a potential aptamer sequence that binds a target, and
I²4 is a primer sequence.

14. The composition of claim 13, wherein H3 is a bead.

15. The composition of claim 13, wherein I¹3 and I²3 are each about 20 nucleotides to about 40 nucleotides long.

16. The composition of claim 13, wherein H2, I¹2, and I²2 are each comprised of a bifunctional linker.

17. The composition of claim 11, wherein H2, I¹2, and I²2 are each comprised of Spacer 18.

18. A composition, the composition comprising three components: a bridge construct, an aptamer construct, and an epitope binding agent construct, wherein
a) the bridge construct comprises
H1-H2-H3;
b) the aptamer construct comprises
I1-I2-I3-I4;
c) the epitope binding agent construct comprises
J1-J2-J3
wherein
H1 is a single-stranded nucleic acid comprising a binding site for I1 and J1,
H2 is a linker that joins H1 and H3,
H3 is a solid support,
I1 is a single-stranded nucleic acid that binds to a complementary region on H1, such that when I3 and J3 bind to a target molecule, I1 stably binds to H1, but in the absence of a target molecule, I1 does not stably bind to H1,
I2 is a linker that joins I1 to I3,
I3 is a potential aptamer sequence that binds a target,
I4 is a primer sequence,
J1 is a single-stranded nucleic acid that binds to a complementary region on H1, such that when I3 and J3 bind to a target molecule, J1 stably binds to H1, but in the absence of a target molecule, J1 does not stably bind to H1,
J2 is a linker that joins J1 to J3, and
J3 is an epitope binding agent that binds to a target.

19. The composition of claim 18, wherein H3 is a bead.

20. The composition of claim 18, wherein I3 is about 20 nucleotides to about 40 nucleotides long.

21. The composition of claim 1 claim 18, wherein H2, I2, and J2 are each comprised of a bifunctional linker.

22. The composition of claim 4 claim 21, wherein H2, I2, and J2 are each comprised of Spacer 18.

23. The composition of claim 1 claim 18, wherein J3 is an antibody or antibody fragment.

24. The composition of claim 1 claim 18, wherein J3 is an aptamer.

25. A method of selecting an aptamer, the method comprising contacting a composition of claim 18 with a target in a reaction mixture and under conditions suitable for creating a stable complex, separating a stable complex of the composition and target from the reaction mixture, and identifying the aptamer(s) that bound the target.

26. The composition of claim 13, wherein
(i) the bridge construct comprises A and B1-B2-B3, such that
A corresponds to H1,
B1 and B2 correspond to H2, and
B3 corresponds to H3;
(ii) the first aptamer construct comprises C¹1-C¹2-C¹3 and D¹1-D¹2-D¹3, such that
C¹1 corresponds to I¹1,
C¹2, C¹2 and D¹1 correspond to I¹2,
D¹2 corresponds to I¹3, and
D¹3 corresponds to I¹4; and
(iii) the second aptamer construct comprises C²1-C²2-C²3 and D²1-D²2-D²3, such that
C²1 corresponds to I²1,
C²2, C²2 and D²1 correspond to I²2,
D²2 corresponds to I²3, and
D²3 corresponds to I²4; and
wherein
A is a single-stranded nucleic acid comprising a binding site for B1, C¹1, and C²1,
B1 is a single-stranded nucleic acid that binds to a complementary region on A,
B2 is a linker that joins B1 to B3,
B3 is a solid support,
C¹1 is a single-stranded nucleic acid that binds to a complementary region on A, such that when D¹2 and D²2 bind to a target molecule, C¹1 stably binds to A, but in the absence of a target molecule, C¹1 does not stably bind to A,
C¹2 is a linker that joins C¹1 to C¹3,
C¹3 is a single-stranded nucleic acid that is complementary to D¹1,
D¹1 is a single-stranded nucleic acid that is complementary to C¹3,
D¹2 is a potential aptamer sequence that binds a target,
D¹3 is a primer sequence,
C²1 is a single-stranded nucleic acid that binds to a complementary region on A, such that when D¹2 and D²2 bind to a target molecule, C²1 stably binds to A, but in the absence of a target molecule, C²1 does not stably bind to A,
C²2 is a linker that joins C²1 to C²3,
C²3 is a single-stranded nucleic acid that is complementary to D²1,
D²1 is a single-stranded nucleic acid that is complementary to C²3,
D²2 is a potential aptamer sequence that binds a target, and
D²3 is a primer sequence.

27. The composition of claim 18, wherein
(i) the bridge construct comprises A and B1-B2-B3, such that
A corresponds to H1,
B1 and B2 correspond to H2, and
B3 corresponds to H3;
(ii) the aptamer construct comprises C¹1-C¹2-C¹3 and D¹1-D¹2-D¹3, such that
C1 corresponds to I1,
C2, C2 and D1 correspond to I2,
D2 corresponds to I3, and
D3 corresponds to I4; and
(iii) the epitope binding agent comprises E1-ES-E3 and F1-F2-F3, such that
E1 corresponds to J1,
E2, E3, F1, and F2 correspond to J2, and
F3 corresponds to J3; and
wherein
A is a single-stranded nucleic acid comprising a binding site for B1, C1, and E1,
B1 is a single-stranded nucleic acid that binds to a complementary region on A,
B2 is optionally a linker that joins B1 to B3,
B3 is optionally a solid support,
C1 is a single-stranded nucleic acid that binds to a complementary region on A, such that when D2 and F3 bind to a target molecule, C1 stably binds to A, but in the absence of a target molecule, C1 does not stably bind to A,
C2 is a linker that joins C1 to C3,
C3 is a single-stranded nucleic acid that is complementary to D1,
D1 is a single-stranded nucleic acid that is complementary to C3,
D2 is a potential aptamer sequence that binds a target,
D3 is a primer sequence,
E1 is a single-stranded nucleic acid that binds to a complementary region on A, such that when D2 and F3 bind to a target molecule, E1 stably binds to A, but in the absence of a target molecule, E1 does not stably bind to A,
E2 is a linker that joins E1 to E3,
E3 is a single-stranded nucleic acid that is complementary to F1.
F1 is a single-stranded nucleic acid that is complementary to E3,
F2 is a linker that joins F1 and F3, and
F3 is an epitope binding agent that binds to a target.


SEQUENCE LISTING (42 sequences)

SEQ ID NO: 1 - DNA, 76 bp, Artificial Sequence
  based on Homo sapiens
ctgtcgttagtgaaggttggcaacaacgaccgggactaacggcagcagcgacaccggtaacgccatatcacagacg

SEQ ID NO: 2 - DNA, 76 bp, Artificial Sequence
  based on Homo sapiens
ctgtcgttagtgaaggttggtaacaacgaccgggactaacggcagcagcgacaccggtaacgccatatcacagacg

SEQ ID NO: 3 - DNA, 75 bp, Artificial Sequence
  based on Homo sapiens
ctgtcgttagtgaaggttggcggcggaccggaacgagcggcagtcgcagtcgaccccaacgccatatcacagacg

SEQ ID NO: 4 - DNA, 75 bp, Artificial Sequence
  based on Homo sapiens
ctgtcgttagtgaaggttgccaagcgaactgcgacaggggatccaggagacggccccaacgccatatcacagacg

SEQ ID NO: 5 - DNA, 76 bp, Artificial Sequence; identical to SEQ ID NO: 1
  based on Homo sapiens

SEQ ID NO: 6 - DNA, 75 bp, Artificial Sequence
  based on Homo sapiens
ctgtcgttagtgaaggttgcaggcacgctgcgatatgtccagttgacccggtttgccaacgccatatcacagacg

SEQ ID NO: 7 - DNA, 75 bp, Artificial Sequence
  based on Homo sapiens
ctgtcgttagtgaaggttggcggcggaccggaactagcggcagtcacagtcgaccccaacgccatatcacagacg

SEQ ID NO: 8 - DNA, 75 bp, Artificial Sequence
  based on Homo sapiens
ctgtcgttagtgaaggttgccccgggaaccgaaacttgcgacctgtcgtcaccttgtaacgccatatcacagacg

SEQ ID NO: 9 - DNA, 74 bp, Artificial Sequence
  based on Homo sapiens
ctgtcgttagtgaaggttggcggcggaccggaacgaacggcagcagcgacaccggtaacgccatatcacagacg

SEQ ID NO: 10 - DNA, 76 bp, Artificial Sequence; identical to SEQ ID NO: 1
  based on Homo sapiens

SEQ ID NO: 11 - DNA, 75 bp, Artificial Sequence; identical to SEQ ID NO: 6
  based on Homo sapiens

SEQ ID NO: 12 - DNA, 75 bp, Artificial Sequence
  based on Homo sapiens
ctgtcgttagtgaaggttggcgcaggaacctgaacgttcgacggggtcggcacctgtaacgccatatcacagacg

SEQ ID NO: 13 - DNA, 76 bp, Artificial Sequence; identical to SEQ ID NO: 1
  based on Homo sapiens

SEQ ID NO: 14 - DNA, 75 bp, Artificial Sequence; identical to SEQ ID NO: 12
  based on Homo sapiens

SEQ ID NO: 15 - DNA, 68 bp, Artificial Sequence
  based on Homo sapiens
ctgtcgttagtgaaggttgccgcccaacgccatatcacagacggccgcccaacgccatancacagacg

SEQ ID NO: 16 - DNA, 66 bp, Artificial Sequence
  based on Homo sapiens
taggtgctcgacgctgacccagacccaccaatatatgccccgcggtgtactcagtcgcaggtcatg

SEQ ID NO: 17 - DNA, 66 bp, Artificial Sequence
  based on Homo sapiens
taggtgctcgacgctgacccaggacggcaccttaaccgcgcgtgggctactcagtcgcaggtcatg

SEQ ID NO: 18 - DNA, 66 bp, Artificial Sequence
  based on Homo sapiens
taggtgctcgacgctgacccaggggacacctatcaatcgtcgtgcggtactcagtcgcaggtcatg

SEQ ID NO: 19 - DNA, 66 bp, Artificial Sequence
  based on Homo sapiens
taggtgctcgacgctgacgggcgacaccgagggggctggggcgcgggtactcagtcgcaggtcatg

SEQ ID NO: 20 - DNA, 66 bp, Artificial Sequence
  based on Homo sapiens
taggtgctcgacgctgacccagggctacaccttatccattcgcgcggtactcagtcgcaggtcatg

SEQ ID NO: 21 - DNA, 66 bp, Artificial Sequence
  based on Homo sapiens
taggtgctcgacgctgacccaatcctgacacaccgtattaagccgcgtactcagtcgcaggtcatg

SEQ ID NO: 22 - DNA, 66 bp, Artificial Sequence
  based on Homo sapiens
taggtgctcgacgctgacccacgaaagacaccgcagtccctccacgctactcagtcgcaggtcatg

SEQ ID NO: 23 - DNA, 66 bp, Artificial Sequence
  based on Homo sapiens
taggtgctcgacgctgaccccccagtcacaccttatgaccgtcccgccactcagtcgcaggtcatg

SEQ ID NO: 24 - DNA, 66 bp, Artificial Sequence
  based on Homo sapiens
taggtgctcgacgctgacccaggcacacctatccaactgtcaccggccactcagtcgcaggtcatg

SEQ ID NO: 25 - DNA, 66 bp, Artificial Sequence
  based on Homo sapiens
taggtgctcgacgctgaccgggcattgacaccttatcgtggggtggggactcagtcgcaggtcatg

SEQ ID NO: 26 - DNA, 42 bp, Artificial Sequence
  based on Homo sapiens
ataaggtgtcgactggattagtaggtgctgcacgctgacaaa

SEQ ID NO: 27 - DNA, 33 bp, Artificial Sequence
  based on Homo sapiens
tttttttttttttttgtcagcgtgcagcaccta

SEQ ID NO: 28 - DNA, 33 bp, Artificial Sequence; identical to SEQ ID NO: 27
  based on Homo sapiens

SEQ ID NO: 29 - DNA, 32 bp, Artificial Sequence
  based on Homo sapiens
gtcagcgtcgagcacctattttgtttccagtc

SEQ ID NO: 30 - DNA, 32 bp, Artificial Sequence
  based on Homo sapiens
gacacctatgttttcgtctgtgatatggcgtt

SEQ ID NO: 31 - DNA, 18 bp, Artificial Sequence
  based on Homo sapiens
taggtgctcgacgctgac

SEQ ID NO: 32 - DNA, 18 bp, Artificial Sequence
  based on Homo sapiens
gtcagcgtcgagcaccta

SEQ ID NO: 33 - DNA, 75 bp, Artificial Sequence
  based on Homo sapiens
ctgtcgttagtgaaggttnnnnnnnnnnnnnnnnnnnnnnnnnnnnnnnnnnnnnnnaacgccatatcacagacg

SEQ ID NO: 34 - DNA, 18 bp, Artificial Sequence
  based on Homo sapiens
ctgtcgttagtgaaggtt

SEQ ID NO: 35 - DNA, 18 bp, Artificial Sequence
  based on Homo sapiens
cgtctgtgatatggcgtt

SEQ ID NO: 36 - DNA, 66 bp, Artificial Sequence
  based on Homo sapiens
taggtgctcgacgctgacnnnnnnnnnnnnnnnnnnnnnnnnnnnnnnactcagtcgcaggtcatg

SEQ ID NO: 37 - DNA, 18 bp, Artificial Sequence
  based on Homo sapiens
taggtgctcgacgctgac

SEQ ID NO: 38 - DNA, 18 bp, Artificial Sequence
  based on Homo sapiens
catgacctgcgactgagt

SEQ ID NO: 39 - DNA, 58 bp, Artificial Sequence
  based on Homo sapiens
aaaggtgtcgactcgttttctggagcgactggattagttttaggtgctgcacgctgac

SEQ ID NO: 40 - DNA, 39 bp, Artificial Sequence
  based on Homo sapiens
acgagtcgacacctttgttttcgtctgtgatatggcgtt

SEQ ID NO: 41 - DNA, 39 bp, Artificial Sequence
  based on Homo sapiens
aaccttcactaacgacagttttgtatccagtcgctccag

SEQ ID NO: 42 - DNA, 39 bp, Artificial Sequence
  based on Homo sapiens
atccagtcgctccagtgttttcgtctgtgatatggcgtt